(19) United States
(12) Patent Application Publication
(10) Pub. No.: US 2025/0090903 A1
(43) Pub. Date: Mar. 20, 2025

(54) RANGING BEACON SYSTEM

(71) Applicant: Garmin International, Inc., Olathe, KS (US)

(72) Inventors: Jason D. Howe, Olathe, KS (US); Zachary A. Deibert, Olathe, KS (US); Nicolas E. Dinsmore, Olathe, KS (US); Thedric D. Jones, Cary, NC (US); David F. Lammers-Meis, Olathe, KS (US); Andre Froncioni, Cochrane (CA)

(21) Appl. No.: 18/882,173

(22) Filed: Sep. 11, 2024

Related U.S. Application Data
(60) Provisional application No. 63/582,712, filed on Sep. 14, 2023.

(51) Int. Cl. A63B 24/00; A63B 71/06; G01S 1/04; G01S 1/68

(52) U.S. Cl. CPC A63B 24/0062; A63B 71/0622; G01S 1/0428; G01S 1/68; A63B 2071/0625; A63B 2220/836; A63B 2225/20; A63B 2225/50; A63B 2225/74

(57) ABSTRACT

A ranging beacon system to measure the performance of a running athlete can include a ranging base and a wearable device. The ranging base can be placed along an exercise path with line of sight to an athlete and can include a ranging base ultra-wideband (UWB) transceiver. The ranging base UWB transceiver can receive a first UWB signal and transmit a second UWB signal. The wearable device can be worn by the athlete and the wearable device can include a wearable device UWB transceiver, and a processor. The wearable device UWB transceiver can transmit the first UWB signal and receive the second UWB signal. The processor can generate ranging data of the athlete based on the first and the second UWB signals.

### RELATED APPLICATIONS

This application claims priority to U.S. provisional application 63/582,712 filed Sep. 14, 2023, the contents of which are hereby incorporated by reference herein for all purposes.

### TECHNICAL FIELD

The present disclosure relates generally to a ranging beacon system for a running athlete.

### BACKGROUND

Smartwatches and other fitness devices such as bike computers are often used to record athletic activities. These devices typically rely on global navigation satellite systems (GNSS) like global positioning systems (GPS) to calculate speed and distance.

### BRIEF DESCRIPTION OF THE DRAWINGS

The detailed description references the accompanying figures. The use of the same reference numbers in different instances in the description and the figures may indicate similar or identical items. In addition, the proportion and the relative scale of the elements provided in the figures are intended to illustrate various embodiments of the present disclosure and are not to be used in a limiting sense.

FIG. 1 illustrates an example of an athlete using a ranging base.

FIG. 2A is a block hardware diagram of a ranging base.

FIG. 2B is a view of a ranging base.

FIG. 3 illustrates an example of an athlete using a ranging beacon system.

FIG. 4A is a block hardware diagram of a wearable device.

FIG. 4B is a view of a wearable device.

FIG. 4C is a front view of a wearable device.

FIG. 5 is a front view of a belt and a wearable device.

FIG. 6 is a block hardware diagram of a ranging beacon system.

FIG. 7 is a table of configurations for a ranging beacon system.

FIG. 8 illustrates an example of an athlete using a ranging beacon system.

FIG. 9 is a block hardware diagram of a computing device.

FIG. 10 is a block diagram of an ecosystem including a wearable device and ranging base.

FIG. 11A illustrates an example of a user interface of a computing device.

FIG. 11B illustrates an example of a user interface of a computing device.

FIG. 11C illustrates an example of a user interface of a computing device.

FIG. 11D illustrates an example of a user interface of a computing device.

FIG. 11E illustrates an example of a user interface of a computing device.

FIG. 11F illustrates an example of a user interface of a computing device.

FIG. 11G illustrates an example of a user interface of a computing device.

FIG. 11H illustrates an example of a user interface of a computing device.

FIG. 11I illustrates an example of a user interface of a computing device.

FIG. 11J illustrates an example of a user interface of a computing device.

FIG. 11K illustrates an example of a user interface of a computing device.

FIG. 11L illustrates an example of a user interface of a computing device.

FIG. 12 is a table of ranging beacon system activities.

FIG. 13 is a block diagram of ranging beacon system solutions.

FIG. 14 illustrates an example of a ranging base position.

FIG. 15 is a graph of time versus radial distance from a ranging base during a 30-meter sprint.

FIG. 16 is a graph of time versus radial distance from a ranging base during a 30-meter sprint and a quadratic curve.

FIG. 17 illustrates an example of possible locations of a wearable device.

FIG. 18 illustrates an example positioning of four ranging bases.

### DETAILED DESCRIPTION

The present disclosure includes a ranging beacon system to measure the performance of a running athlete. The ranging beacon system can include a ranging base and a wearable device. The ranging base is configured to be placed along an exercise path with line of sight to an athlete and can include a ranging base ultra-wideband (UWB) transceiver. The ranging base UWB transceiver can be configured to receive a first UWB signal and to transmit a second UWB signal. The wearable device can be configured to be worn by the athlete and the wearable device can include a wearable device UWB transceiver and a processor. The wearable device UWB transceiver can be configured to transmit the first UWB signal and receive the second UWB signal. The processor can be coupled to the wearable device UWB transceiver. The processor can be configured to generate ranging data of the athlete based on the first and the second UWB signals. The ranging data can include distance, position, and time.

Conventional fitness devices can rely on GNSS, which works well in many situations, but can struggle to generate accurate results where precise finish times are necessary. For example, when timing a short distance sprint where inches and milliseconds matter. Further, timing devices can require multiple costly units that can provide time, but not speed. In contrast, UWB is a radio technology that uses a very low energy level for short-range, high-bandwidth communications over a large portion of the radio spectrum (e.g., greater than 500 megahertz). UWB can be used for real-time positioning due to its precision and reliability.

The ranging beacon system disclosed herein is configured to provide accurate metrics for athletes including time and speed data. The ranging beacon system can work in combination with a wearable device. In some configurations, the wearable device can be a smartwatch that can provide (e.g., transmit) additional data including personal data of the athlete.

In various embodiments, the ranging beacon system can further include a computing device. The computing device can be a smartphone configured to receive the ranging data and/or the personal data of the athlete and display the ranging data and/or the personal data on a user interface of the computing device. The system, the base(s), and the wearable device(s) can transmit personal data of the athlete to other devices, such as the athlete's smartphone, using Bluetooth and/or other communication techniques.

In some embodiments, the wearable device can be configured to provide real-time feedback to the athlete based on performance metrics obtained during a sprint activity. The feedback can be delivered via audio cues through an integrated speaker, visual cues through the user interface or LEDs, or haptic feedback through vibrations generated by the wearable device. For example, the device can alert the athlete to increase stride length, maintain acceleration, or improve cadence based on the detected metrics, such as stride frequency, acceleration, or deceleration. This real-time coaching capability can enable the athlete to make immediate adjustments to their performance, improving training efficiency and effectiveness. The feedback can be tailored to the specific goals of the athlete, such as achieving optimal acceleration during the start phase or maintaining maximum speed throughout the sprint.

In a number of embodiments, the ranging beacon system can be configured to precisely measure the athlete's reaction time from a starting signal to initial movement. The starting signal can be an auditory cue from a speaker, a visual cue from an LED, or a tactile cue from a vibrating component integrated into the wearable device. The processor of the wearable device can be configured to detect the exact moment the athlete begins to move, using data from sensors such as accelerometers, gyroscopes, or UWB transceivers. The reaction time can then be calculated by determining the time difference between the starting signal and the first detected motion. By analyzing reaction times over multiple trials, the system can identify patterns or trends and provide targeted recommendations to the athlete to optimize their start phase, such as improving response speed to auditory cues or refining body positioning in the starting stance.

Further, the wearable device and/or ranging base can be configured to monitor the athlete's gait dynamics in real-time during a sprint activity. Metrics such as foot placement, ground contact time, vertical oscillation, stride length, and torso angle can be continuously recorded and analyzed by the processor to determine the efficiency of the athlete's running technique. Based on the collected data, the system can detect inefficiencies, such as excessive vertical motion or improper foot strike patterns, which may result in energy loss or reduced speed. The wearable device can then provide dynamic feedback, such as audible alerts, visual signals, or haptic vibrations, to guide the athlete in adjusting their technique. This feedback can be aimed at optimizing factors like stride length and foot placement to enhance performance, helping the athlete maintain optimal form and speed throughout the sprint.

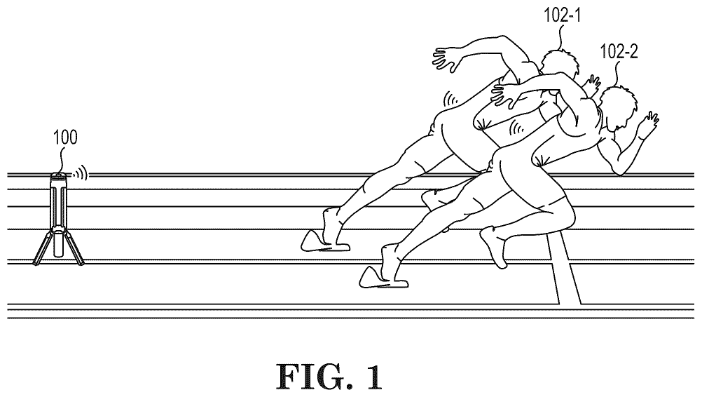

FIG. 1 illustrates an example of a first athlete 102-1 and a second athlete 102-2 using a ranging base 100 to record metrics. The ranging base 100 can work in combination with a wearable device or a computing device or function as a stand-alone device without requiring pairing or interaction with other devices.

In a number of embodiments, the ranging base 100 may work in combination with one or more other ranging bases and/or one or more wearable devices. The number of ranging bases including ranging base 100 can be positioned at stationary locations while the wearable devices are worn or carried by athletes 102-1, 102-2. In some examples, the ranging base 100 may be positioned before or on a start line (e.g., behind the start line from the perspective of the athletes 102-1, 102-2) or on or after a finish line (e.g., beyond the finish line from the perspective of the athletes 102-1, 102-2). In a number of embodiments, ranging base 100 along with another ranging base can be positioned before the start line. Ranging base 100 can record metrics of the first athlete 102-1 while the other ranging base can record metrics of the second athlete 102-2. However, each ranging base 100 may be used by any number of athletes, so for instance two or more athletes may use the same ranging base 100 at the same time (or even at different times) to record activities.

The ranging base 100 and a wearable device may utilize UWB for positioning and timing calculations without relying on GNSS. The ranging base 100 may be configured as a pylon, an anchor, or any other stationary object suitable for placement on the ground along an exercise path. The exercise path can be, but is not limited to, a track or a pool, for example.

The wearable device may be configured to be strapped to a user, clipped to a user's bib or clothing, and/or integrated into other devices such as a smartwatch, heart rate monitor, or running pod. Utilization of a wearable device mounted near the user's center, such as on the user's waist or torso, eases timing calculations performed by the ranging beacon system. However, wrist-mounted wearables, such as those incorporated into smartwatches, may be utilized by accounting for the user's arm motion while running.

In some configurations, the ranging base 100 may be the user's smartphone and the wearable device may be the user's smartwatch, so that custom wearable devices and ranging bases 100 are not required to generate the timing information described herein. For instance, an athlete 102 can place their smartphone at or behind the start line, enable a timing activity on his or her smartwatch, and then run (sprint, bike, swim, jump, etc.) to record accurate ranging data. In a number of embodiments, the wearable device may be omitted entirely and two ranging bases 100 may be used. For example, one ranging base 100 can be at the start and a second ranging base 100 can be at the finish. Similarly, ranging data may be calculated by any portion of the system and/or by combinations of components of the system. data,

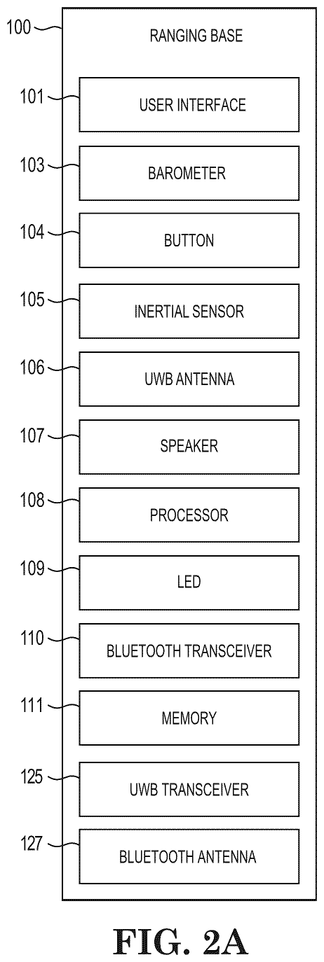

FIG. 2A is a block hardware diagram of a ranging base 100. The ranging base 100 can include a user interface 101, a barometer 103, one or more buttons 104, an inertial sensor 105, a UWB antenna 106, a speaker 107, a processor 108, one or more light-emitting diodes (LEDs) 109, a Bluetooth transceiver 110, a memory 111, a UWB transceiver 125 (that may include or otherwise be integrated with UWB antenna 106), and/or a Bluetooth antenna 127.

The processor 108 provides processing functionality for the ranging base 100 and can include any number of processors, micro-controllers, circuitry, field programmable gate array (FPGA) or other processing systems, and resident or external memory for storing data, executable code, and other information. The processor 108 is not limited to being formed from any particular material or the processing mechanisms employed therein and, as such, can be implemented via semiconductor(s) and/or transistors (e.g., using electronic integrated circuit (IC) components), and so forth.

The processor 108 can execute one or more software programs embodied in a non-transitory computer readable medium (e.g., memory 111) that implement techniques described herein including transmitting and receiving the UWB signal via the UWB transceiver 125, generating ranging data of the athlete based on the UWB signal, and transmitting the ranging data via the Bluetooth transceiver 110. The ranging data may include any information derived at least in part from the timing of the transmitted and/or received UWB signals. In a number of embodiments, the ranging base 100 can also transmit an additional UWB signal to an additional wearable device via the UWB transceiver 125. The wearable device itself may calculate ranging data in addition to or as an alternative to the ranging data calculated by the base 100. Further, the processor 108 can be configured to communicate (e.g., receive and/or transmit data) with a different ranging base via the UWB transceiver 125.

The memory 111 can be a tangible, computer-readable storage medium that provides storage functionality to store various data and/or program code associated with an operation, such as software programs and/or code segments, or other data to instruct the processor 108, and possibly other components of the ranging base 100, to perform the functionality described herein. The memory 111 can store data, such as program instructions for operating the ranging base 100 including its components, and so forth. The memory 111 can also store metrics including run time, speed, acceleration, reaction time, force-velocity, step rate, step index, torso angle, step count, step length, leg turnover, ground contact time (GCT), flight time, vertical oscillation, step speed loss, maximum force (Fmax), and/or spinout speed. The memory 111 can further store these metrics for a number of athletes or for a number of performances.

It should be noted that while a single memory 111 is described, a wide variety of types and combinations of memory (e.g., tangible, non-transitory memory) can be employed. The memory 111 can be integral with the processor 108, can comprise stand-alone memory, or can be a combination of both. Some examples of the memory 111 can include removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory devices, hard disk memory, external memory, and so forth. In a number of embodiments, the ranging base 100 and/or the memory 111 can include removable integrated circuit card (ICC) memory, such as memory provided by a subscriber identity module (SIM) card, a universal subscriber identity module (USIM) card, a universal integrated circuit card (UICC), and so on.

The barometer 103 can determine atmospheric pressure. In some examples, the barometer 103 can be used in conjunction with a barometer of a wearable device to estimate a cosine effect. The cosine effect can be a difference between a measured radial distance to the athlete and a horizontal distance. The barometer 103 can also be used to determine whether the athlete is running up or down hill.

The one or more buttons 104 can be configured to cause the ranging base 100 to be paired with other devices (e.g., by operation of the processor 108, Bluetooth transceiver 110, and/or other components of the ranging base 100). For example, a user can press a button of the one or more buttons 104 to pair the ranging base 100 to a wearable device and/or a computing device. The speaker 107 can broadcast an audible cue and/or the one or more LEDs 109 can emit light to indicate a Bluetooth connection and/or a UWB connection. Further, the speaker 107 can broadcast an audible cue and/or the one or more LEDs 109 can emit light to indicate a start of a race. In some examples, a number of ranging bases, including ranging base 100, can be placed around a track to act as timing gates. Each ranging base can broadcast an audible cue and/or emit light to indicate to the athlete whether they are behind or ahead of a target pace.

The inertial sensor 105 can be coupled to the processor 108. An orientation of the ranging base 100 can be sensed by the inertial sensor 105. The ranging base 100 should be placed along an exercise path with line of sight to an athlete. If a ranging base 100 is tipped over and/or has no line of sight to the athlete, the ranging base 100 may not be able to transmit the UWB signal to the athlete and/or receive a UWB signal from the athlete. Failure to transmit or receive a UWB signal to and/or from the athlete could cause the processor 108 to fail to generate ranging data of the athlete or generate inaccurate ranging data of the athlete.

The processor 108 can determine whether the ranging base 100 is tipped over (e.g., based on input from the inertial sensor 105) and/or has no line of sight to the athlete (e.g., based on input from the UWB antenna 106). The speaker 107 can broadcast an audible cue and/or the one or more LEDs 109 can emit light to indicate the ranging base 100 is tipped over and/or has no line of sight to the athlete. In a number of embodiments, the Bluetooth transceiver 110 can transmit a notification in response to the processor 108 determining that the ranging base 100 is tipped over and/or has no line of sight to the athlete. Once the ranging base 100 is in a proper orientation, the speaker 107 can be used to broadcast an audible cue and/or the one or more LEDs 109 can emit light to indicate a start of a race.

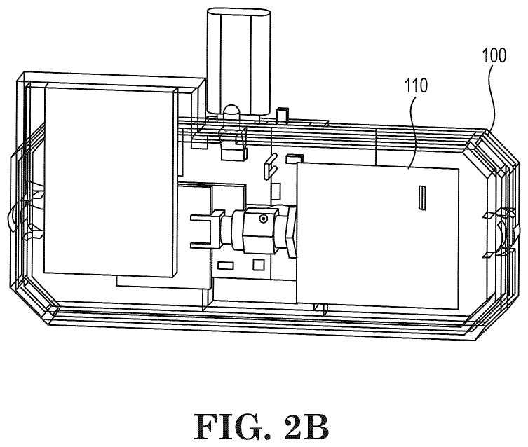

The UWB transceiver 125 can transmit a first UWB signal to the athlete. A second UWB signal can then be received by the UWB transceiver 125 from the athlete. More particularly, the first UWB signal can be transmitted from the UWB transceiver 125 of the ranging base 100 to a device of the athlete (e.g., wearable device 112 illustrated in FIG. 3) and the device can transmit a second UWB signal back to the ranging base 100. Timing and/or position data of the athlete can be generated based on the UWB signals by the processor 108 coupled to the UWB transceiver 125. For example, the processor 108 can be configured to calculate ranging data including timing and/or position data based on time of flight, time difference of arrival, two way ranging, or other techniques known in the art. The use of the terms “first” and “second” with respect to the UWB signals does not limit the UWB signals to being transmitted in any particular order. Rather, the terms “first” and “second” are used to differentiate the signals. In some embodiments, the ranging base 100 can operate as a UWB radar without the need for a corresponding device being worn by the athlete. In some examples, the UWB transceiver 125 can be configured to transmit the ranging data to other devices via UWB.

The ranging base 100 can also include a user interface 101. The user interface 101 can be coupled to processor 108 and can be configured to display ranging data. For example, the ranging base 100 can display the athlete's time and/or other metrics after the athlete has finished the activity. This enables the athlete and/or coach to view and/or record the time and/or other metrics.

FIG. 2B is a view of a ranging base 100. The ranging base 100 can include the Bluetooth transceiver 110. The Bluetooth transceiver 110 coupled to the processor 108 can transmit the ranging data for use and/or additional calculations by other devices. In some examples, the ranging base 100 can receive data via the the Bluetooth transceiver 110 including Bluetooth antenna 127.

FIG. 3 illustrates an example of an athlete 102 using a ranging beacon system. The ranging beacon system can include a ranging base 100 and a wearable device 112. The ranging base 100 can be placed at or before a start line, as illustrated in FIG. 3. In a number of embodiments, a different ranging base 100 can be positioned at or after a finish line.

The wearable device 112 can be a watch, an activity band, or, as illustrated in FIG. 3, a tag. The wearable device 112 can fasten to a garment or a belt via a clip, magnet, hook and loop fasteners, molded inserts/sleeves, combinations thereof, and the like.

In a number of embodiments, the wearable device 112 can send data to the ranging base 100 then the ranging base 100 can send the data back to the wearable device 112 along with some additional data. The wearable device 112 can calculate a round-trip time it took for the data to be sent to the ranging base 100 then received back at the wearable device 112. From this time, the wearable device 112 can derive a distance (e.g., a range) between the wearable device 112 and the ranging base 100.

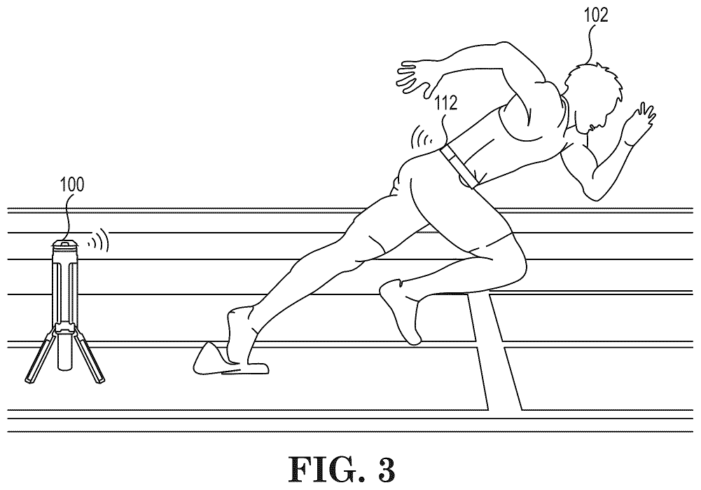

FIG. 4A is a block hardware diagram of a wearable device 112. The wearable device 112 can include one or more LEDs 113, a UWB antenna 114, a speaker 115, a processor 116, a memory 117, a Bluetooth transceiver 118, a user interface 119, an inertial sensor 123, a barometer 156, a UWB transceiver 129 (which may include or incorporate UWB antenna 114), a Bluetooth antenna 131, and/or one or more buttons 133.

The UWB transceiver 129, using UWB antenna 114, can receive the first UWB signal from the ranging base and transmit the second UWB signal back to the ranging base via the UWB transceiver 129 or the UWB transceiver 129 can transmit the first UWB signal to the ranging base and receive the second UWB signal back from the ranging base. In a number of embodiments, an additional wearable device can receive a third UWB signal from the ranging base and transmit a fourth UWB signal to the ranging base or transmit the third UWB signal to the ranging base and receive the fourth UWB signal from the ranging base.

The processor 116 provides processing functionality for the wearable device 112 and can include any number of processors, micro-controllers, circuitry, field programmable gate array (FPGA) or other processing systems, and resident or external memory for storing data, executable code, and other information. The processor 116 is not limited by the materials from which it is formed or the processing mechanisms employed therein and, as such, can be implemented via semiconductor(s) and/or transistors (e.g., using electronic integrated circuit (IC) components), and so forth. The processor 116 can execute one or more software programs embodied in a non-transitory computer readable medium (e.g., memory 117) that implement techniques described herein including receiving ranging data from the ranging base (e.g., time, range, and/or speed information corresponding to the user's activity) and displaying the ranging data via a user interface 119. In some examples, the wearable device 112 can transmit data via the Bluetooth transceiver 118.

Processor 116 of wearable device 112 may additionally or alternatively calculate ranging data from UWB signals transmitted and received by UWB transceiver 129 without relying on base 100 for such calculations. For example, processor 116 can measure time of flight directly without requiring ranging base 100 to make this calculation and transmit it to wearable device 112. Similarly, ranging data may be calculated partially by both base 100 and wearable device 112 so each device may independently, or in combination, provide the functionality described herein.

The memory 117 can be a tangible, computer-readable storage medium that provides storage functionality to store various data and/or program code associated with an operation, such as software programs and/or code segments, or other data to instruct the processor 116, and possibly other components of the wearable device 112, to perform the functionality described herein. The memory 117 can store data, such as program instructions for operating the wearable device 112 including its components, and so forth. The memory 117 can also store metrics including run time, speed, acceleration, reaction time, force-velocity, step rate, step index, torso angle, step count, step length, leg turnover, ground contact time (GCT), flight time, vertical oscillation, step speed loss, maximum force (Fmax), and/or spinout speed. The memory 117 can further store personal data. Personal data can include, but is not limited to age, weight, and height of the athlete.

It should be noted that while a single memory 117 is described, a wide variety of types and combinations of memory (e.g., tangible, non-transitory memory) can be employed. The memory 117 can be integral with the processor 116, can comprise stand-alone memory, or can be a combination of both. Some examples of the memory 117 can include removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory devices, hard disk memory, external memory, and so forth. In a number of embodiments, the wearable device 112 and/or the memory 117 can include removable integrated circuit card (ICC) memory, such as memory provided by a subscriber identity module (SIM) card, a universal subscriber identity module (USIM) card, a universal integrated circuit card (UICC), and so on.

The processor 116 can determine the metrics including the run time, speed, acceleration, reaction time, force-velocity, step rate, step index, torso angle, step count, step length, leg turnover, ground contact time (GCT), flight time, vertical oscillation, step speed loss, maximum force (Fmax), and/or spinout speed based on the ranging data. The memory 117 can further store machine-readable instructions for implementing the user interface 119 to allow a user to input commands to, and receive information from, the wearable device 112.

The user interface 119 can be displayed on a liquid crystal display (LCD), a thin film transistor (TFT), a light-emitting diode (LED), a light-emitting polymer (LEP), and/or a polymer light-emitting diode (PLED) capable of presenting text, graphical, and/or pictorial information. The user interface 119 may be backlit such that it may be viewed in the dark or other low-light environments. One example embodiment is a 100-pixel by 64-pixel film compensated super-twisted nematic display (FSTN) including a bright white light-emitting diode (LED) backlight. However, in a number of embodiments, the wearable device 112 does not include a display and a user can input commands to and/or display information from the wearable device 112 via a computing device, for example.

The wearable device 112 may include a transparent lens that covers and/or protects components of the wearable device 112. The wearable device 112 may be provided with a touch screen to receive input (e.g., data, commands, etc.) from an athlete and/or coach. For example, an athlete may operate the wearable device 112 by touching the touch screen and/or by performing gestures on the screen. In some embodiments, the touch screen may be a capacitive touch screen, a resistive touch screen, an infrared touch screen, combinations thereof, and the like.

The wearable device 112 may further include one or more input/output (I/O) devices (e.g., a keypad, buttons, a wireless input device, a thumbwheel input device, etc.). The I/O devices may include one or more audio I/O devices, such as a microphone, speakers, and the like. Additionally, user input may be provided from movement of the wearable device 112. For example, the inertial sensor 123 (e.g., accelerometer) may be used to identify vertical, horizontal, angular movement, and/or tapping of the wearable device 112 or the lens.

In accordance with one or more embodiments of the present disclosure, the user interface 119 may include one or more buttons 133 to control a function of the wearable device 112. The one or more buttons 133 can also be used to pair the wearable device 112 to the UWB of the ranging base or initiate a sprinting activity, for example. Functions of the wearable device 112 may be associated with a location determining component and/or a performance monitoring component. Functions of the wearable device 112 may include, but are not limited to, displaying a current geographic location of the wearable device 112, mapping a location on the user interface 119, locating a desired location and displaying the desired location on the user interface 119, and presenting information based on ranging data, a physiological characteristic (e.g., heart-rate (HR), heart-rate variability, blood pressure, or SpO2 percentage, for example), and/or a physiological response (e.g., stress level, body energy level, etc.) of the athlete.

In some examples, the user interface 119 is configured to display the run time, speed, acceleration, reaction time, force-velocity, step rate, step index, torso angle, step count, step length, leg turnover, ground contact time (GCT), flight time, vertical oscillation, step speed loss, maximum force (Fmax), and/or spinout speed based on the ranging data.

In some examples, UWB time-of-flight (ToF) functionality can be used to calculate the ranging data described herein. For instance, the wearable device 112 and/or the ranging base can use ToF information to determine relative distances between the wearable device 112 and the ranging base. In some examples, ToF information may be converted into distance information. In other examples, ToF information may be directly used by the wearable device 112 and/or ranging base to determine distance information or otherwise identify when the athlete has begun movement and/or crossed the finish line. For instance, a decrease in distance then an increase indicates that the wearable device 112 has moved past the ranging base. Likewise, a decrease in ToF then an increase in ToF indicates that the wearable device 112 has crossed the ranging base. Similar changes in distance and/or ToF can be used to determine the start of movement and the end of movement.

In a number of embodiments, the wearable device 112 and/or ranging base may be configured to determine the relative angle to another wearable device 112 and/or ranging base. For instance, the ranging base can calculate an angle to the wearable device 112 by computing the phase difference of arrival between two antennas incorporated within the ranging base. This measurement can be used to look for the point where the phase difference of arrival (PDoA) is zero, assuming the two antennas are perpendicular to the finish line. This could be used independently or in conjunction with the ToF or distance methods to calculate timing information.

In a number of embodiments, the speaker 115 can broadcast an audible cue and/or the one or more LEDs 113 can emit light to indicate a Bluetooth connection and/or a UWB connection. Further, the speaker 115 can broadcast an audible cue and/or the one or more LEDs 113 can emit light to indicate a start of a race and/or a sprinting activity.

Although not illustrated in FIG. 4A, the wearable device 112 can further include a number of sensors. For example, the wearable device 112 can include an accelerometer, a gyroscope, a GNSS, and/or a heart rate sensor. When ranging data is merged with metrics such as GPS coordinates from the wearable device 112 and accelerometer readings, a multidimensional dataset can be formed. This dataset provides information about the athlete's motion, orientation, speed, and physiological response, such as heart rate. By synchronizing data from various sources, the ranging beacon system can offer accurate tracking, particularly in locations where GPS signals might be inconsistent. In areas like dense urban environments or inside buildings, the combined data from the ranging base and other sensors can supplement any missing or weak GPS data, ensuring a continuous and accurate representation of the athlete's movement.

The wearable device 112 being able to house multiple sensors such as barometer 156, an accelerometer, gyroscope, GNSS, and/or heart rate sensor allows it to operate as an independent monitoring tool. When an athlete is in motion, these sensors can record nuanced details about their performance. For example, the accelerometer and gyroscope can capture changes in speed and movement dynamics, while the heart rate sensor can provide insights into the athlete's cardiovascular response to the activity. With an integrated display on the wearable device 112 and/or the ranging base, the athlete can receive real-time feedback or post-activity analysis, which can be instrumental for training adjustments and performance optimization. This data may additionally or alternatively be displayed by other devices, such as the user's mobile phone or other computing devices. This consolidated data aids in forming a detailed understanding of an athlete's performance, facilitating informed decisions on training strategies and techniques. For instance, the athlete can look at his or her watch immediately after completing an activity (such as a spring) to receive accurate feedback.

In the context of a 100-meter dash, accurate data provides a quantitative foundation for understanding an athlete's performance metrics. By merging ranging data from the ranging beacon system with metrics such as accelerometer readings and heart rate information, a precise timeline of the athlete's progression over the 100 meters can be established. This includes details like the time taken to achieve peak velocity, consistency of speed throughout the dash, and any fluctuations in acceleration or deceleration. Additionally, heart rate data can give insights into the athlete's cardiovascular exertion at different phases of the sprint, indicating the body's physiological response at various points along the 100 meters.

Having this granularity in data offers practical applications for training and strategy. For instance, the UWB combined with accelerometer information can identify specific phases in the sprint where the athlete might be losing momentum or not achieving optimal acceleration. This can guide targeted training interventions, such as specific drills to improve the start phase or the final push. Simultaneously, heart rate data can suggest how the athlete's stamina and energy expenditure relate to their speed, helping coaches devise tailored conditioning programs or pacing strategies.

In the 100-meter dash, the start is a critical phase. Using data collected, such as ranging data from the ranging beacon system and accelerometer readings from integrated sensors, a comprehensive picture of the athlete's initial acceleration and push-off from the blocks can be drawn. This data can specifically indicate the force exerted during the initial push, the time taken to transition from a crouched position to full stride, and the sequence of footfalls in the first few meters. These metrics provide a basis for analyzing the efficiency of an athlete's start technique.

Precise timing information is important in analyzing the correlation between an athlete's gait and their overall performance in the 100-meter dash. By closely examining the timestamps associated with each footfall, coaches and analysts can determine the exact duration between strides. This can reveal insights into stride frequency and how consistently it's maintained throughout the race. If, for instance, the time intervals between footfalls vary significantly at certain portions of the dash, it might suggest a disruption in the athlete's rhythm or a momentary lapse in form. Similarly, comparing segments of the sprint, like the first 50 meters to the last 50 meters, with the associated ranging data can help identify if and when an athlete's gait becomes less efficient, potentially due to fatigue or technique breakdown. This precise timing information, when juxtaposed against overall race time and other performance metrics, allows for a deeper understanding of how gait influences speed and endurance, thereby directing targeted improvements in training and strategy.

The barometer 156 can determine atmospheric pressure. In some examples, the barometer 156 can be used in conjunction with a barometer of a ranging base (e.g., barometer 103 of FIG. 2A) to estimate a cosine effect. The barometer 156 can also be used to determine whether the athlete is running up or down hill. FIG. 4B is a view of a wearable device 112. The wearable device 112 can include a UWB antenna 114 along with attachment portions 120-1, 120-2 for fastening the wearable device 112 to the athlete. In the example illustrated in FIG. 4B, the attachment portions 120-1, 120-2 are lugs configured to removably attach the wearable device 112 to the athlete with belts, straps, or clips, for example.

Although not shown, the wearable device 112 can further include an optical assembly, including one or more emitters (e.g., LEDs) of visible and/or non-visible light and one or more receivers (e.g., photodiodes) of visible and/or non-visible light that generate a light intensity signal based on the received reflection of light. The wearable device 112 can determine cardiac information, such as heart rate (HR) and pulse oximetry information based on the light intensity signal.

FIG. 4C is a front view of a wearable device 112. The attachment portion 120 illustrated in FIG. 4C can be a clip. The attachment portion 120 can fasten to a belt or waistband of the athlete, for example. A portion of the belt or waistband can be clamped between the main body 121 of the wearable device 112 and the attachment portion 120.

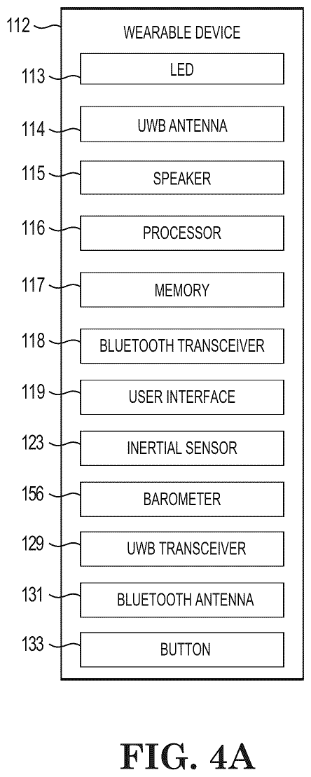

FIG. 5 is a front view of a belt 122 and a wearable device 112. The belt 122 can be worn by the athlete. For example, the belt 122 can be fastened around the hips or the torso of the athlete. As illustrated in FIG. 5, the belt 122 can be clamped between the main body 121 of the wearable device 112 and the attachment portion 120 of the wearable device 112.

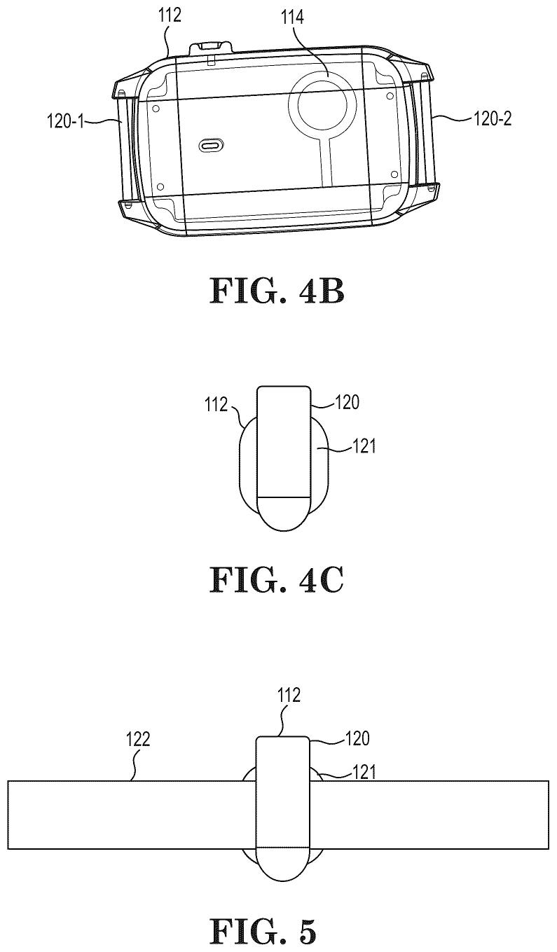

FIG. 6 is a block hardware diagram of a ranging beacon system. The ranging beacon system can include an inertial measurement unit (IMU) 124, a magnetic sensor 132, a System on Chip (SoC) 144, and a non-volatile memory 152. The ranging beacon system can further include optional components, which can be indicated in FIG. 6 via dashed boxes.

The wearable device can include an IMU 124. The IMU 124 can be an always-on 3-axis accelerometer and 3-axis gyroscope. The IMU 124, when combined with the UWB or other distance measurements can determine speed and movement of the athlete.

One or more buttons 126 can be included in the wearable device. The one or more buttons 126 can be configured to cause the wearable device to be paired with other devices. For example, a user can press the one or more buttons 126 to pair the wearable device to the ranging base and/or a computing device.

As previously described in connection with FIG. 4A, the wearable device can include a speaker 115. The speaker 115 can broadcast an audible cue to indicate a Bluetooth connection and/or a UWB connection. Further, speaker 115 can broadcast an audible cue to indicate a start of a race.

A power management integrated circuit (PMIC) 130 can be included in the wearable device and/or the ranging base. The PMIC 130 can control the flow of power in the wearable device and/or the ranging base.

The wearable device can include a magnetic sensor 132. The magnetic sensor 132 can determine the magnetic field for the x, y, and z axes to determine an orientation of the wearable device in space.

As previously described in connection with FIG. 4A, the wearable device can include one or more LEDs 113. The one or more LEDs 113 can emit light to indicate a Bluetooth connection and/or a UWB connection. Further, the one or more LEDs 113 can emit light to indicate a start of a race. In some examples, the one or more LEDs 113 can emit light to indicate whether the wearable device is turned on or off.

Both the wearable device and the ranging base can include a RF transceiver 146, such as a UWB transceiver (including an UWB antenna 136 like those disclosed in the preceding paragraphs. The UWB transceiver can be used to transmit and/or receive UWB signals. The UWB signals can be used to generate ranging data of the athlete by the ranging base, wearable device, other computing devices, and/or combinations thereof.

Battery 138 can be included in the wearable device. The wearable device can be powered by the battery 138. Battery 148 can be included in the ranging base and can be used to power the ranging base. Both the wearable device and the ranging base can include a charger 140. Battery 138 and/or battery 148 can be charged using charger 140. However, in some examples, battery 138 and/or battery 148 can be a non-rechargeable replaceable battery.

A connector 142 can be included in the wearable device and/or the ranging base. The connector 142 can couple the charger 140, the battery 138, and/or the battery 148 to a SoC 144.

The SoC 144 can include Flash and RAM and can include protocol support for Bluetooth Low Energy (LE). The SoC 144 can be included on the wearable device and/or the ranging base. A near-field communication (NFC) coil 150 can be included in the wearable device. The NFC coil 150 can be used for low-power wireless communication. The NFC coil 150 can pair a wearable device to a ranging base and/or associate a wearable device with a user, a belt, or a computing device.

Both the wearable device and the ranging base can include a radio frequency (RF) transceiver 146. The RF transceiver 146 can transmit and receive radio waves using an antenna.

Both the wearable device and the ranging base can include a non-volatile memory 152. The non-volatile memory 152 can be flash memory. For example, the non-volatile memory 152 can be 128 MB flash memory.

The wearable device and/or the ranging base can include a Bluetooth antenna 154, which can correspond to Bluetooth antenna 127 of FIG. 2A and/or Bluetooth antenna 131 of FIG. 4A. The Bluetooth antenna 154 can be a 2.4 GHZ antenna.

The wearable device can include a barometer 156. The barometer 156 can determine barometric pressure. Relative changes in altitude can be determined by changes in barometric pressure or the difference in pressure at the wearable device versus the ranging base. In some examples, the barometer 156 can be used in conjunction with a barometer of a ranging base to estimate a cosine effect. The barometer 156 can also be used to determine whether the athlete is running up or down hill.

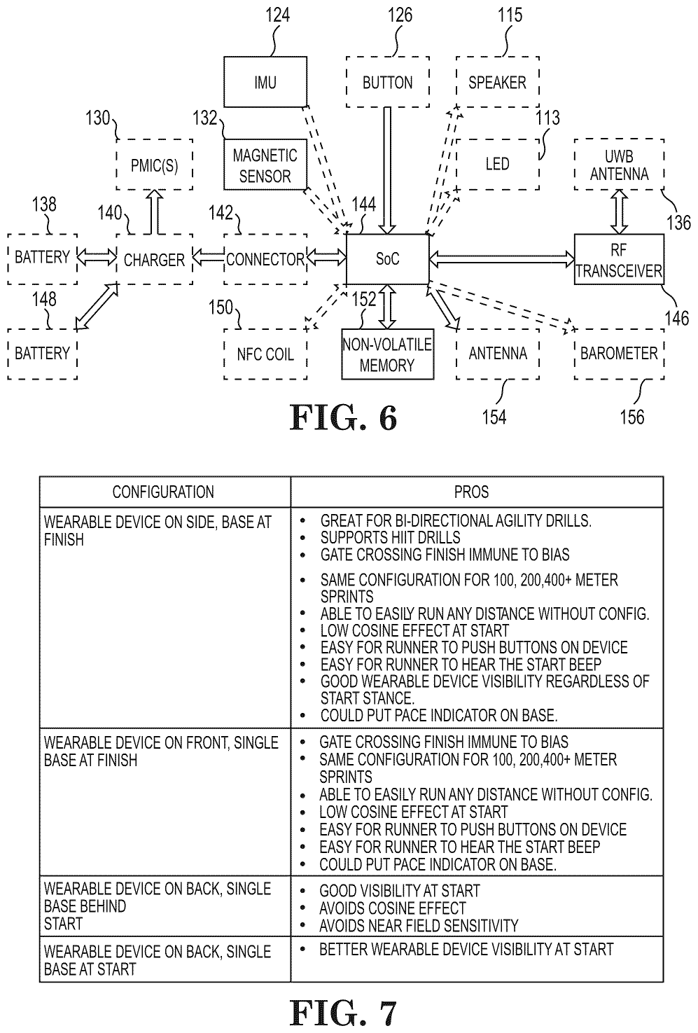

FIG. 7 is a table of configurations for a ranging beacon system. The wearable device can be located on a side of the athlete while the ranging base is at the finish line. This configuration is great for bi-directional agility drills, supports high-intensity interval training (HIIT) drills, and is immune to bias during a gate crossing finish. The wearable device on the side of the athlete and the base at the finish line can be used for the 100-meter sprint, the 200-meter sprint, the 400-meter sprint, and greater distance sprints. The athlete is able to easily run any distance without needing to reconfigure the location of the ranging base. Since the wearable device is positioned at the finish line, any cosine error is low at the start line. Cosine error is also referred to as cosine effect. The athlete can easily push buttons and/or hear the starting cue from the wearable device on the athlete's side. Regardless of the starting stance of the athlete, the wearable device has good visibility to the ranging base including for crouched starts, standing starts, or 3-point starts. Further, a pace indicator could be put on the ranging base in this configuration.

The wearable device can be on the front of the athlete and a ranging base can be at the finish line. This configuration can be immune to bias during a gate crossing finish. The same configuration can be used for the 100-meter sprint, the 200-meter sprint, the 400-meter sprint, and greater distance sprints. The athlete is able to easily run any distance without needing to reconfigure the location of the ranging base. Since the wearable device is positioned at the finish line, any cosine error is low at the start line. The athlete can easily push buttons and/or hear the starting cue from the wearable device on the athlete's front. Further, a pace indicator could be put on the ranging base in this configuration.

The wearable device can be positioned on the back of the athlete and a ranging base behind the start line. In this configuration, there is good visibility at the start because there is no physical obstruction between the wearable device and the ranging base. Further, the wearable device being further from the ranging base even when the athlete is at the start line avoids the cosine effect and near field sensitivity. Any cosine error is negligible due to the low or zero angle between the ranging base and the wearable device, particularly in a straight-line run.

Another configuration can include the wearable device worn on the back of the athlete and the ranging base located at the start line. This configuration provides better visibility to the wearable device at the start and the athlete does not need to go to the finish line to setup the ranging base or to verify that the wearable device is paired with the ranging base.

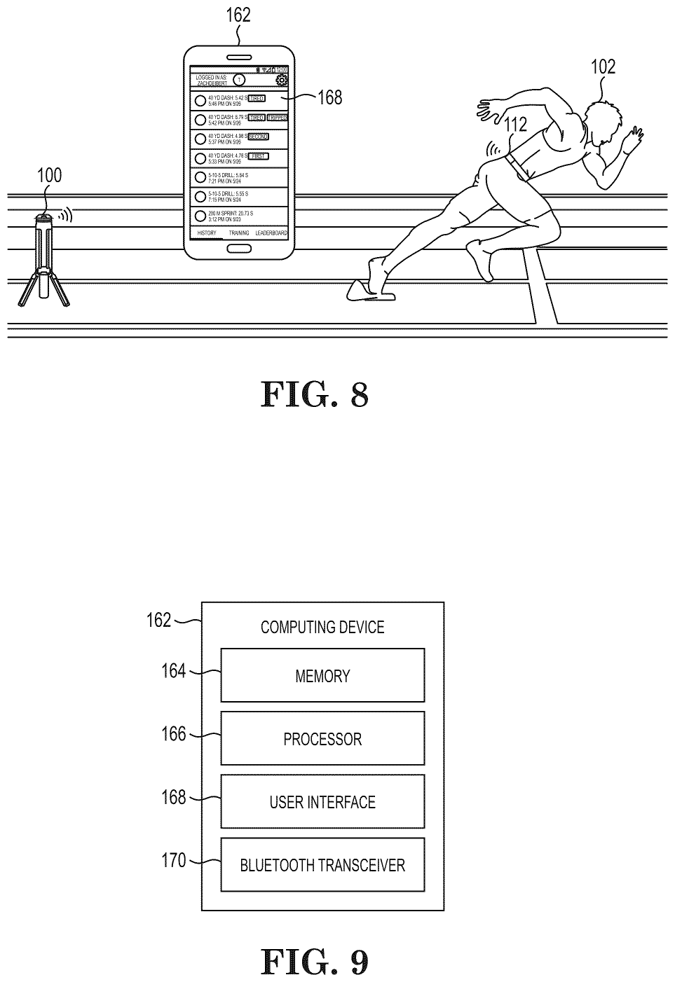

FIG. 8 illustrates an example of an athlete 102 using a ranging beacon system. The ranging beacon system can include the ranging base 100, a computing device 162, and a wearable device 112.

The ranging beacon system configuration illustrated in FIG. 8 depicts the ranging base 100 located behind the start line and the wearable device worn on the back of the athlete as the athlete is running the race and/or sprint activity. The benefits of this configuration were previously described in connection with FIG. 7.

The race and/or sprint activity can be started in several ways including a gun start, an athlete-initiated start, and a flying start. For a gun start, the race timer can start when the starting gun goes off. For these cases, the ranging beacon system could integrate with the starting gun to determine when it went off and start the timer. The computing device 162, the wearable device 112, and/or the ranging base 100 could also provide a starting cue to the athlete to start the race, so they can practice starts without the need for an actual starting gun. Regardless of whether the starting cue is from external sources or generated internally by the ranging beacon system, the starting cue can start the race timer.

When the athlete self-starts the run, the timer can start when the athlete begins moving. This can be detected automatically by looking for the UWB distance to the finish line to begin decreasing, looking for the UWB distance to increase if the athlete is in front of the ranging base 100, and/or looking for acceleration on the accelerometer. The timer starts when this movement is detected. A button on the ground that the athlete presses when they are ready and releases when they begin to run could also be used. The timer can start when the button is released.

For a flying start, the athlete can start behind the start line and accelerate before crossing the start line. The timer can start when the athlete crosses the start line defined by the distance of the run. For example, for a 30-meter flying sprint, the start time will start when the athlete is 30 meters from the start line as measured by the UWB. Since the UWB knows the distance the athlete is from the finish line, there is no need to have any electronics at the actual starting line.

When the race or sprint activity is over, the computing device 162 can display the ranging data on a user interface 168. In a number of embodiments, the computing device 162 can be a smartphone, a tablet, a laptop, a watch, or a desktop computer.

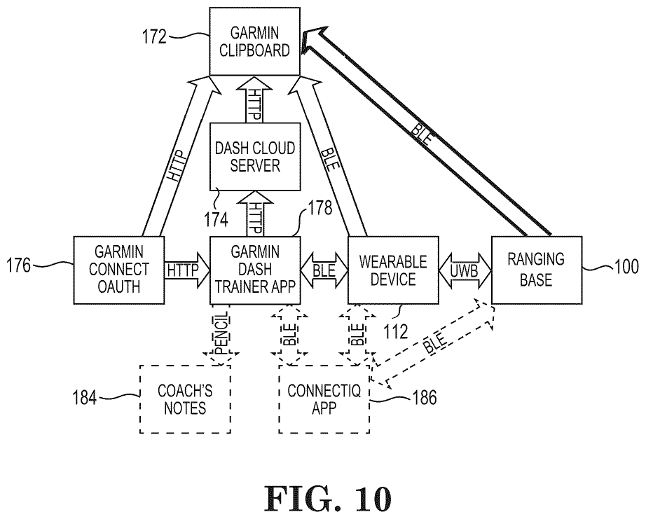

FIG. 9 is a block hardware diagram of a computing device 162. The processor 166 provides processing functionality for the computing device 162 and can include any number of processors, micro-controllers, circuitry, field programmable gate array (FPGA) or other processing systems, and resident or external memory for storing data, executable code, and other information. The processor 166 is not limited by the materials from which it is formed or the processing mechanisms employed therein and, as such, can be implemented via semiconductor(s) and/or transistors (e.g., using electronic integrated circuit (IC) components), and so forth. The processor 166 can execute one or more software programs embodied in a non-transitory computer readable medium (e.g., memory 164) that implement techniques described herein including receiving ranging data via the Bluetooth transceiver 170 and displaying the ranging data via a user interface 168.

The memory 164 can be a tangible, computer-readable storage medium that provides storage functionality to store various data and/or program code associated with an operation, such as software programs and/or code segments, or other data to instruct the processor 166, and possibly other components of the computing device 162, to perform the functionality described herein. The memory 164 can store data, such as program instructions for operating the computing device 162 including its components, and so forth. The memory 164 can also store metrics including run time, speed, acceleration, reaction time, force-velocity, step rate, step index, torso angle, step count, step length, leg turnover, ground contact time (GCT), flight time, vertical oscillation, step speed loss, maximum force (Fmax), and/or spinout speed. The memory 164 can further store personal data. Personal data can include, but is not limited to, age, weight, and height of the athlete.

It should be noted that while a single memory 164 is described, a wide variety of types and combinations of memory (e.g., tangible, non-transitory memory) can be employed. The memory 164 can be integral with the processor 166, can comprise stand-alone memory, or can be a combination of both. Some examples of the memory 164 can include removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory devices, hard disk memory, external memory, and so forth. In a number of embodiments, the computing device 162 and/or the memory 164 can include removable integrated circuit card (ICC) memory, such as memory provided by a subscriber identity module (SIM) card, a universal subscriber identity module (USIM) card, a universal integrated circuit card (UICC), and so on.

The processor 166 can determine the metrics including the run time, speed, acceleration, reaction time, force-velocity, step rate, step index, torso angle, step count, step length, leg turnover, ground contact time (GCT), flight time, vertical oscillation, step speed loss, maximum force (Fmax), and/or spinout speed based on the ranging data. The memory 164 can further store machine-readable instructions for implementing the user interface 168 to allow a user to input commands to, and receive information from, the computing device 162. In some examples, the user interface 168 is configured to display the run time, speed, acceleration, reaction time, force-velocity, step rate, step index, torso angle, step count, step length, leg turnover, ground contact time (GCT), flight time, vertical oscillation, step speed loss, maximum force (Fmax), and/or spinout speed based on the ranging data.

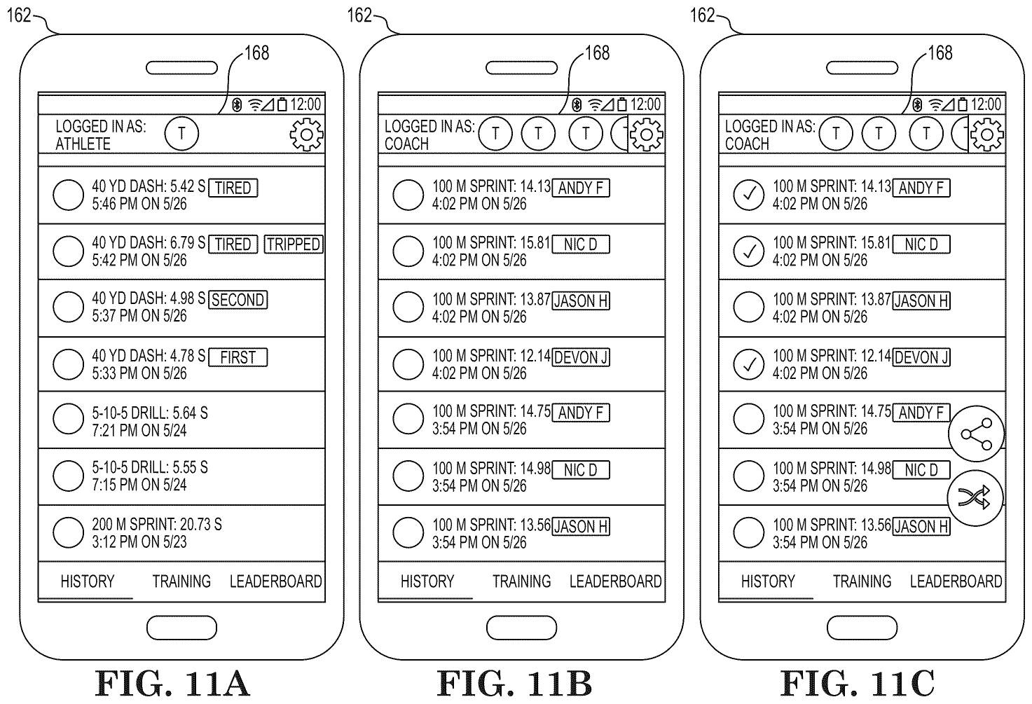

FIG. 10 is a block diagram of an ecosystem including a wearable device 112 and a ranging base 100. The wearable device 112 and/or the ranging base 100 of a ranging beacon system can communicate with an application 178 running on a computing device, which in turn can communicate with other applications and servers to combine and distribute information generated by the ranging beacon system. For example, ranging data generated by the ranging beacon system may be combined with GPS, accelerometer, and heart rate data generated by the user's wearable device 112 and/or computing device for the same activity. In some examples, the wearable device 112 may itself include integrated sensors, including an accelerometer, gyroscope, GNSS, heart rate sensor, temperature sensor, magnetometer, and the like, to generate metrics for the user in addition to ranging data. The wearable device 112 and/or the ranging base 100 may also include an integrated display for displaying information, including calculated metrics, without requiring the use of a separate device.

The ranging base 100 and the wearable device 112 can communicate with each other using UWB and/or Bluetooth Low Energy (BLE). For example, the ranging base 100 can transmit a UWB signal to the wearable device 112. The wearable device 112 can transmit data including ranging data via Bluetooth including BLE to the application 178, which can be the Garmin Dash Trainer application or to a different application 186, which can be the Connect IQ or other mobile application hosted on a watch, for example.

Coach's notes 184 can be taken from application 178. Application 178 can transmit data via Hypertext Transfer Protocol (HTTP) to a server 174, which can be the Dash Cloud server. Server 174 can transmit data to a clipboard 172 via HTTP. The clipboard 172 can be the Garmin Clipboard. An authenticated web service 176, for example, Garmin Connect OAuth can transmit data to the clipboard 172 and/or application 178 via HTTP. In a number of embodiments, the ranging base 100 and/or the wearable device 112 can communicate directly with application 186 or the clipboard 172 via BLE.

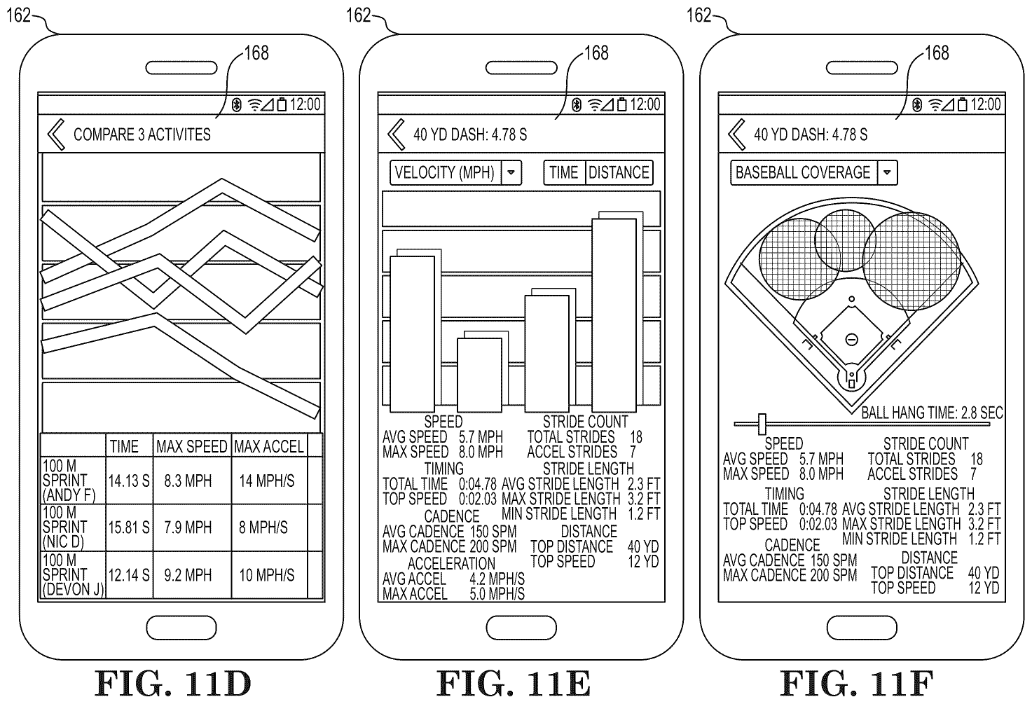

FIG. 11A illustrates an example of a user interface 168 of a computing device 162. The user interface 168 can display an example of the computing device 162 running an application, for example, the Garmin Dash Trainer application. As shown on the user interface 168, an athlete is logged in. Since the athlete is logged in, the user interface 168 only reveals data associated with the athlete. For example, the application provides the athlete's time, the distance, the date, the time, and place for each race or drill under the history section. Further, the application can include details about the athlete including whether they were tired or tripped during the race or drill.

As shown on the user interface 168 of FIG. 11B, a coach is logged in. Since the coach is logged in, the user interface 168 can reveal data associated with a number of athletes. The application can provide times for each athlete that participated in a particular race or drill. In the present example, the coach selected to see the ranging data for the 100-meter sprint for each athlete. The application can show the athlete's time for the 100-meter sprint and the time and date they ran the 100-meter sprint.

As illustrated by the user interface 168 of FIG. 11C, the coach is still logged in. The coach can select, via the user interface 168, the data associated with one or more of the number of athletes. In the present example, the coach has selected Andy F, Nic D, and Devon J, as shown by the check marks in line with their names.

As shown on the user interface 168 of FIG. 11D, ranging data associated with Andy F, Nic D, and Devon J is displayed. The ranging data, in the present example, is displayed as a line chart to compare the three athletes along with their race times, max speeds, and max accelerations in a table.

As illustrated on the user interface 168 of FIG. 11E, ranging data associated with one or more athletes can be displayed as a bar chart. Specifically, velocity over time is displayed in the present example. The user interface 168 can further display an average speed, maximum speed, total strides, acceleration strides, total time, top speed, average stride length, maximum stride length, minimum stride length, average cadence, maximum cadence, top distance, top speed, average acceleration, and maximum acceleration for the 40 yard dash.

As shown on the user interface of FIG. 11F, baseball coverage can be displayed on a baseball field. The baseball coverage can be based on a 40-yard dash time and a ball hang time. In this example the 40-yard dash time is 4.78 seconds and the ball hang time is 2.8 seconds. The ball hang time can be adjusted, which can increase or decrease the baseball coverage of the baseball field. The user interface 168 can further display an average speed, maximum speed, total strides, acceleration strides, total time, top speed, average stride length, maximum stride length, minimum stride length, average cadence, maximum cadence, top distance, and top speed for the 40-yard dash.

As illustrated by the user interface of FIG. 11G, the athlete can choose what they would like to improve at or have the application choose for them under the training section of the application. The athlete can also select a date that they can train up to.

As shown on the user interface of FIG. 11H, the application can provide training suggestions under the training section. In a number of embodiments, the training suggestions can be displayed in order of priority.

As illustrated by the user interface of FIG. 11I, the application can include a leaderboard section. The leaderboard can be filtered by age, area, and/or activity. In the example illustrated in FIG. 11I, the area is Kansas City Metro, the age is from 20 to 29, and the activity is the 40-yard dash. The leaderboard can include the rank, name, and time of each athlete on the leaderboard.

As shown on the user interface of FIG. 11J, an athlete or coach can select a configuration for the ranging beacon system or an activity. The ranging base at the finish line and the wearable on the front of the athlete, the ranging base behind the start line and the wearable device on the back of the athlete, and the ranging base at the finish line and the wearable device on the shoulder are a few example configurations for the ranging beacon system. The 5-10-5 drill, arrowhead drill, and swimming are examples of activities. Once the user selects the configuration for the ranging beacon system or the activity, the coach or athlete can select to start the activity.

As illustrated by the user interface 168 of FIG. 11K, once a coach or athlete selects an activity, the application can provide a description of the activity and instructions on setting up the ranging beacon system. In the present example, the coach or athlete has selected the 5-10-5 drill and the user interface 168 includes a description of the 5-10-5 drill and a picture to show where to set up the ranging base and cones. In some examples, the user interface can further include a setup video.

As shown on the user interface 168 of FIG. 11L, the coach or athlete can select settings regarding privacy, activities, and devices. For example, the coach or athlete can decide whether to participate in leaderboards, show their name, edit attributes, pair a new device, or auto-start all connected wearable devices.

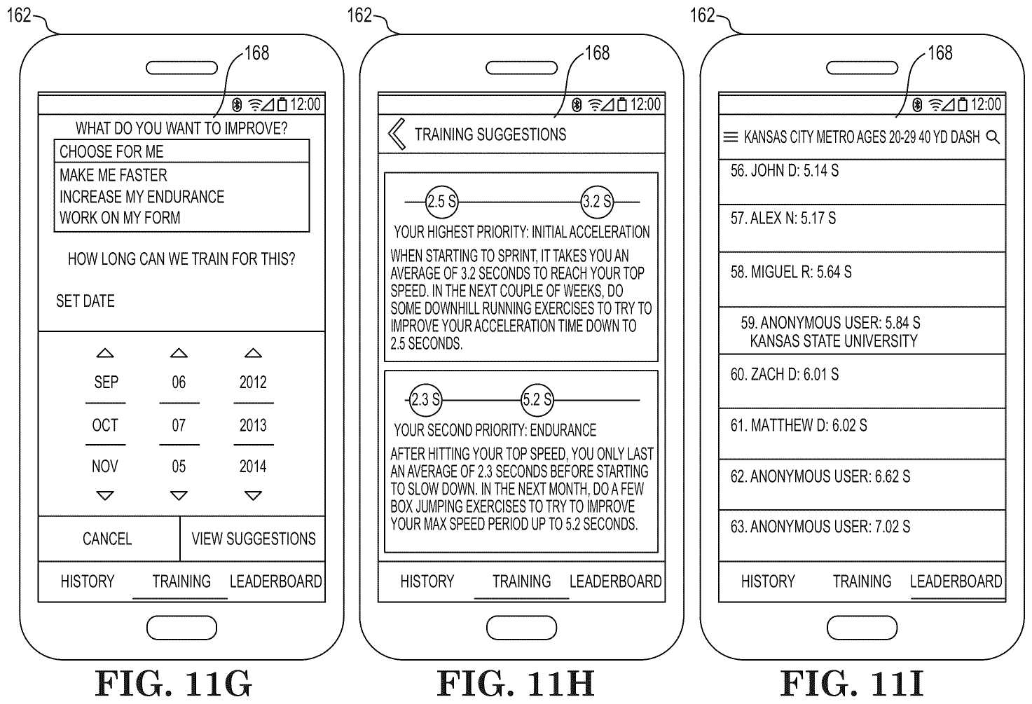

FIG. 12 is a table of ranging beacon system activities. The ranging beacon system can compute metrics for a number of activities that can be relevant for a number of sports including football, baseball, and track & field. For example, metrics for activities like 3 cone drills, agility 5-10-5, box drills, broad jump, circle cone and burst drill, hurdle knee drive, ladder drill, lateral shuffle, and lateral to sprint can be useful metrics for athletes who play football. Basketball players may want the beacon system to compute metrics for bouncing start sprints, falling start sprints, and lateral shuffle.

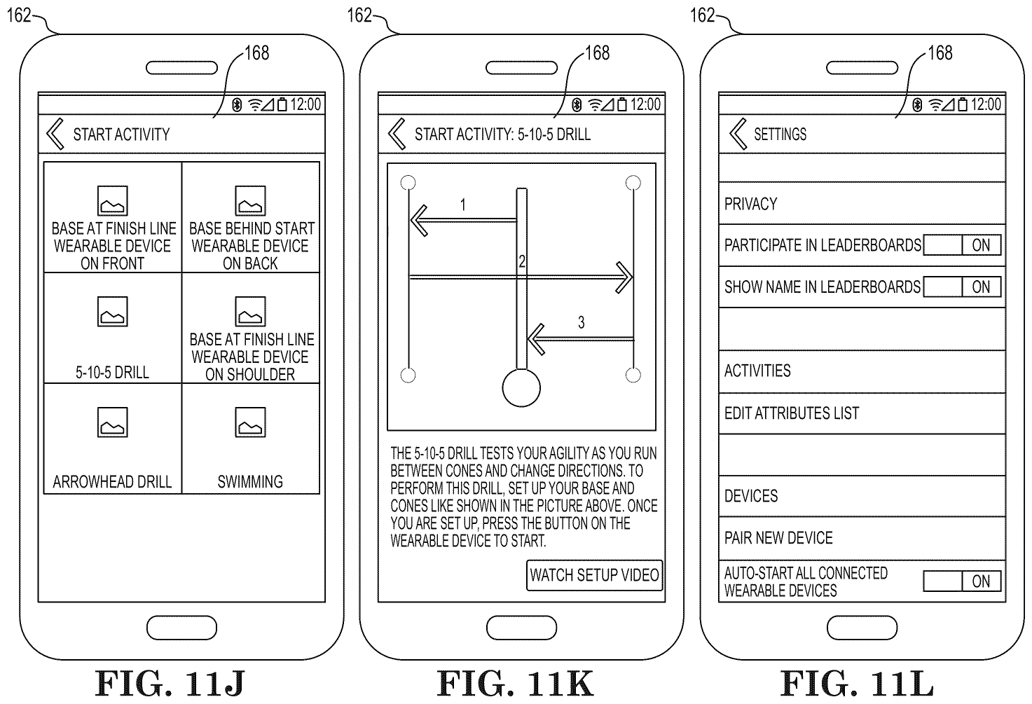

FIG. 13 is a block diagram of ranging beacon system solutions. The ranging beacon system can generate a number of detected metrics 188. These detected metrics can include torso orientation, vertical waist height, braking force, vertical to horizontal ratio, stride length, reaction time, impulse level, vertical acceleration at beginning of step, cycle time, waist twist, and fading force at end of stride, among other detected metrics.

The detected metrics 188 can map to different categories of problems, which can include start problems 190, horizontal phase problems 192, and vertical phase problems 194. Start problems 190 can include low arm swing, lead leg too far forward, curved back, high drive angle, and reaction time. Horizontal phase problems 192 can include flat footed, over-striding, high cycle path, and stride length stall. Vertical phase problems 194 can include tensing up, side-to-side rotation, and inappropriate stride length.

The problems can then be linked to solutions 196 generated by the ranging beacon system. The solutions 196 can include arm swings on command, jump back starts, wall switches, launches, sled, steep hill, three tape drill, sprint bounds, wickets, relax sprint, tempo runs, and leg hops, for example.

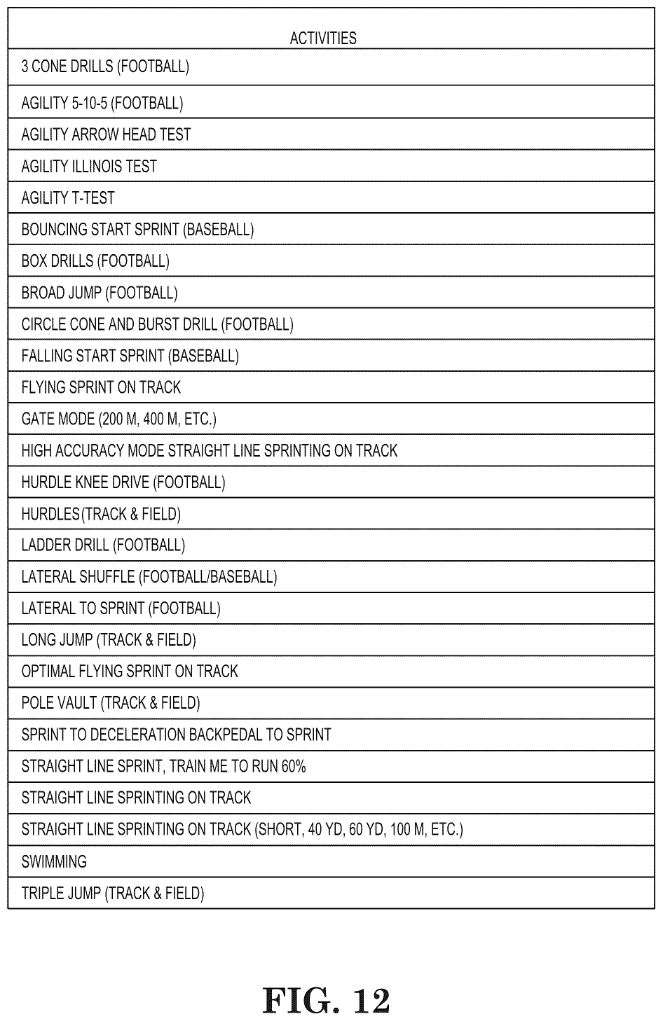

FIG. 14 illustrates an example of a ranging base 100 position. As illustrated in FIG. 14, the ranging base 100 can be located at finish line 202 along the run line 200. In some examples, the ranging base 100 can be located behind the finish line 202, at the start line 198, or ahead of the start line 198. In a number of embodiments, a second ranging base may be positioned at or ahead of the start line 198 when ranging base 100 is positioned at the finish line 202 or behind the finish line 202.

With a single ranging base 100 on the finish line 202, as illustrated in FIG. 14, an assumption can be made that the run line 200 that the athlete is running on is near the ranging base 100. As such, the y distance is small. Since the ranging base 100 measures the radial distance to the wearable device, it is actually measuring \sqrt{x²+y²}. If y is 2 meters and x is 30 meters, the measured distance will be 30.07, so the timer will actually start around 7 centimeters (cm) after the starting line. This error may be small enough not to significantly affect the results, however if the y distance is more than a couple of meters this error becomes more significant. When two ranging bases 100 are used, the actual x distance can be computed using multi-lateration, so the y distance doesn't matter.

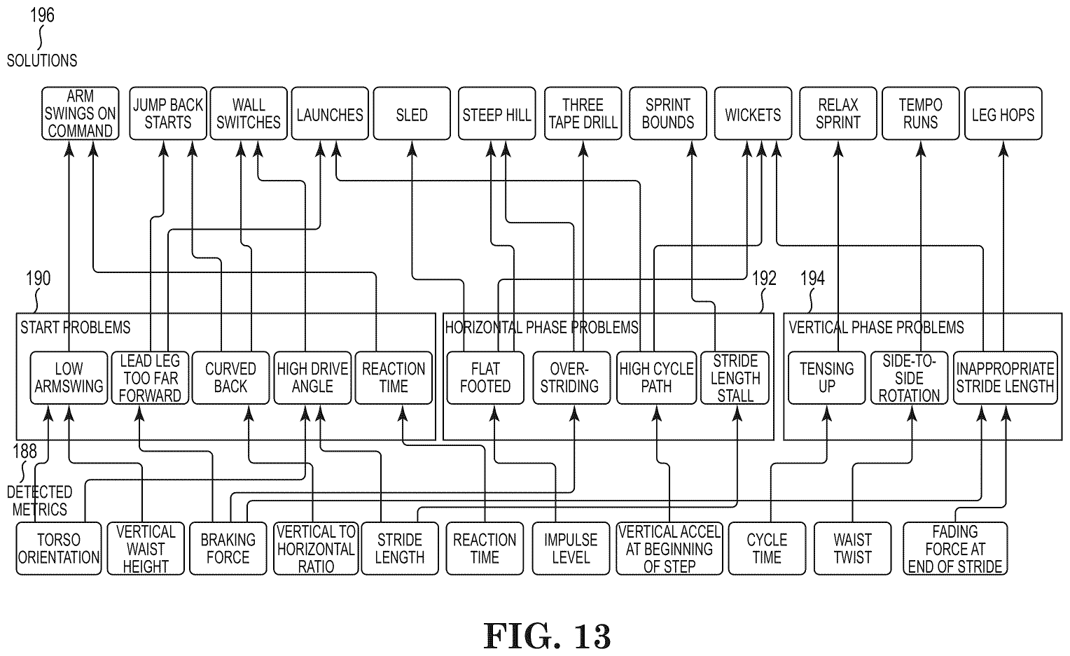

FIG. 15 is a graph of time versus radial distance from a ranging base during a 30-meter sprint. In this example, the ranging base is placed at the finish line. The ranging beacon system can determine when an athlete has passed the finish line crossing 206 by looking for the minimum distance between the wearable device and the ranging base. If the run line is straight, the minimum distance defines the point where the radial distance is perpendicular to the run line which occurs at the finish line. A simple way to process this is to simply look for the minimum distance and call that the finish line crossing 206. The radial distance is pretty straight at the start of the 30-meter 204. The finish line crossing 206 is a V shape as the athlete approaches and then runs away from the finish line crossing 206.

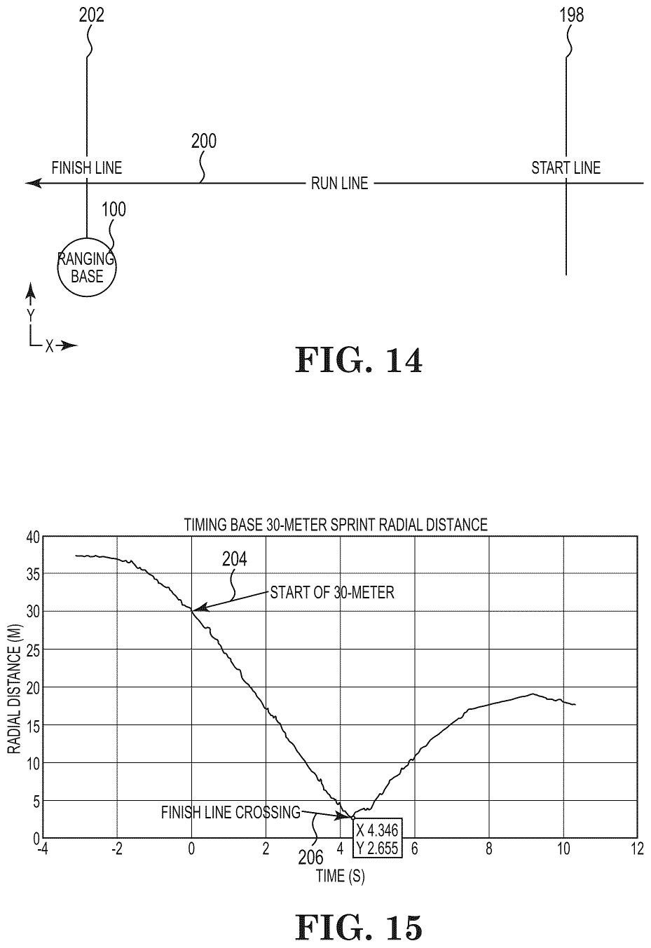

FIG. 16 is a graph of time versus radial distance from a ranging base during a 30-meter sprint and a quadratic curve. The graph of FIG. 16 is zoomed in on the finish line crossing of FIG. 15. A second order fit can be performed to find a more refined minimum distance, which is shown as the quadratic curve.

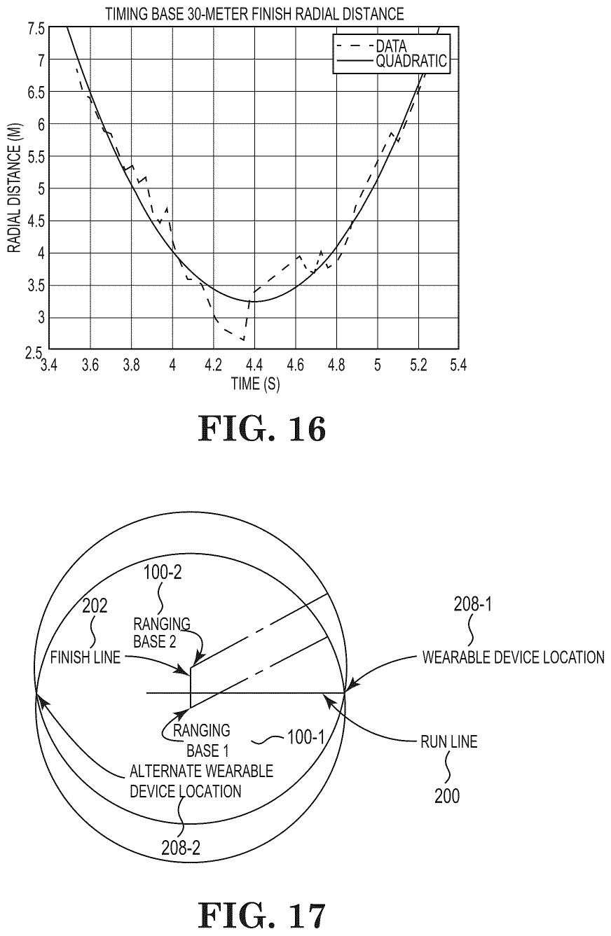

FIG. 17 illustrates an example of possible locations of a wearable device. When two or more ranging bases 100-1, 100-2 are used to define the finish line 202, multi-lateration can be used to find the location of the wearable device. Multi-lateration uses the two or more radial distances from the ranging bases 100-1, 100-2 to the wearable device to find the location in x,y space of the wearable device. As shown in FIG. 17, multi-lateration generates a circle for each ranging base 100-1, 100-2 with the center of the circle at the ranging base location and the radius equal to the radial distance to the wearable device. It then finds the intersection of these circles to determine the possible wearable device locations 208-1, 208-2. With two ranging bases 100-1, 100-2 there is an ambiguity where there are actually two points in space where the wearable device can be. For example, the two circles intersect in two different possible wearable device locations 208-1, 208-2. This is overcome by assuming the athlete starts the run with a positive x value. When the athlete crosses the finish line 202, the x location will appear as a sharp V with the bottom point of the V near zero. The algorithm can use this point to find the zero crossing and use that to stop the timer. The x location makes a V because of the assumption that x is positive, so when the x switches from positive to negative when the athlete crosses the finish line 202, the algorithm will effectively take the absolute value of x. Of course, the wearable device can communicate with any number of ranging bases using its UWB transceiver.

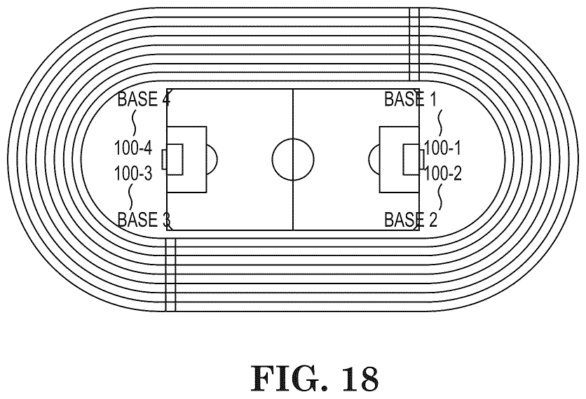

FIG. 18 illustrates an example positioning of four ranging bases 100-1, 100-2, 100-3, 100-4. If an athlete is running on a regulation 400-meter track, it is possible to track their run with a single or a pair of ranging bases if the track orientation is known and their heading is known. For the straight portion of the track, the run is tracked like a 100-meter sprint. For the curved portion of the track, the fact that the athlete turns nearly 180 degrees during the 100 meters can be used to track where they are on the track. For example, if the track is oriented with the straight portions East to West and the athlete's heading is North, then it can be determined that the runner is at the apex of the curve. This along with the UWB range can determine which lane the athlete is in and therefore where they are in the 400-meter sprint.

For distances longer than 100 meters, one or more ranging bases 100-1, 100-2, 100-3, 100-4 can be used to define the start line and a separate set can be used to define the finish line. GPS and/or acceleration data can be used to interpolate velocity in the periods where the wearable device can't see the ranging bases 100-1, 100-2, 100-3, 100-4. In some uses, such as where the user is running an event where the start line is the same as the finish line (e.g., the 400 m), a single ranging base may be utilized.

With GPS, a single set of ranging bases 100-1, 100-2, 100-3, 100-4 can be placed at the start and/or finish line. GPS, acceleration, and knowledge about the shape of the track can be used to fill in the gaps when the wearable device can't see the ranging bases 100-1, 100-2, 100-3, 100-4.

Without GPS, ranging bases 100-1, 100-2, 100-3, 100-4 can be placed around the track to accurately keep track of the athlete's position as they go around the track, as illustrated in FIG. 18. Knowledge of the track shape and accelerometer data can be used to fill in any gaps. As shown in FIG. 18, four ranging bases 100-1, 100-2, 100-3, 100-4 are used to perform multi-lateration to determine the athlete's position on the track.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and methods are used. Therefore, the scope of one or more embodiments of the present disclosure should be determined with reference to the appended claims, along with the full range of equivalents to which such claims are entitled.

As used herein, “a number of” something can refer to one or more of such things. As will be appreciated, elements shown in the various embodiments herein can be added, exchanged, and/or eliminated so as to provide a number of additional embodiments of the present disclosure.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A ranging beacon system to measure a performance of a running athlete, the system comprising:
a ranging base configured to be placed along an exercise path with line of sight to the athlete, wherein the ranging base includes:
a ranging base ultra-wideband (UWB) transceiver configured to:
receive a first UWB signal; and
transmit a second UWB signal; and
a wearable device configured to be worn by the athlete, wherein the wearable device includes:
a wearable device UWB transceiver configured to:
transmit the first UWB signal; and
receive the second UWB signal; and
a processor coupled to the wearable device UWB transceiver, wherein the processor is configured to generate ranging data of the athlete based on the first UWB signal and the second UWB signal.

2. The ranging beacon system of claim 1, wherein the wearable device is a tag, a watch, or an activity band.

3. The ranging beacon system of claim 1, further comprising an additional ranging base comprising:
an additional ranging base UWB transceiver configured to:
receive a third UWB signal; and
transmit a fourth UWB signal.

4. The ranging beacon system of claim 3, further comprising an additional wearable device configured to be worn by an additional athlete comprising:
an additional wearable device UWB transceiver configured to:
transmit the third UWB signal; and
receive the fourth UWB signal.

5. The ranging beacon system of claim 4, wherein the additional wearable device comprises an additional processor coupled to the additional wearable device UWB transceiver, wherein the additional processor is configured to generate ranging data of the additional athlete based on the third UWB signal and the fourth UWB signal.

6. The ranging beacon system of claim 1, further comprising a speaker configured to broadcast an audible cue to indicate a start of a race.

7. The ranging beacon system of claim 1, further comprising one or more light-emitting diodes (LEDs) configured to emit light to indicate a start of a race.

8. The ranging beacon system of claim 1, wherein the processor is configured to communicate with a second ranging base via the wearable UWB transceiver.

9. The ranging beacon system of claim 8, wherein the ranging base is configured to be placed before or on a start line and the second ranging base is positioned on or after a finish line.

10. A ranging beacon system to measure a performance of a running athlete, the system comprising:
a ranging base configured to be placed along an exercise path with line of sight to the athlete, wherein the ranging base includes:
a ranging base ultra-wideband (UWB) transceiver configured to:
transmit a first UWB signal; and
receive a second UWB signal;
a ranging base processor coupled to the ranging base UWB transceiver, wherein the ranging base processor is configured to generate ranging data of the athlete based on the first UWB signal and the second UWB signal; and
a Bluetooth transceiver coupled to the ranging base processor and configured to transmit the ranging data; and
a wearable device, configured to be worn by the athlete, the wearable device comprising:
a Bluetooth transceiver configured to receive the ranging data transmitted by the ranging base; and
a processor configured to determine at least one of speed, acceleration, reaction time, force-velocity, step rate, step index, torso angle, step count, step length, leg turnover, ground contact time (GCT), flight time, vertical oscillation, step speed loss, maximum force (Fmax), and spinout speed based on the ranging data.

11. The ranging beacon system of claim 10, wherein the wearable device is configured to fasten to the athlete via at least one of a clip, magnet, and hook and loop fastener.

12. The ranging beacon system of claim 10, wherein the wearable device further comprises:
a UWB transceiver configured to:
receive the first UWB signal from the ranging base; and
transmit the second UWB signal back to the ranging base.

13. The ranging beacon system of claim 10, wherein the wearable device is configured to transmit personal data of the athlete.

14. The ranging beacon system of claim 10, wherein the ranging base is configured to transmit a third UWB signal via the ranging base UWB transceiver.

15. The ranging beacon system of claim 14, further comprising a second wearable device configured to:
receive the third UWB signal; and
transmit a fourth UWB signal to the ranging base.

16. The ranging beacon system of claim 10, wherein the ranging base further comprises a button configured to pair the ranging base to the wearable device.

17. A ranging beacon system to measure a performance of a running athlete, the system comprising:
a ranging base configured to be placed along an exercise path with line of sight to the athlete, wherein the ranging base includes:
an ultra-wideband (UWB) transceiver configured to:
transmit a first UWB signal; and
receive a second UWB signal; and
a processor coupled to the UWB transceiver, wherein the processor is configured to generate ranging data of the athlete based on the first UWB signal and the second UWB signal, wherein the UWB transceiver is further configured to transmit the ranging data; and
a computing device comprising:
a UWB transceiver configured to receive the ranging data from the ranging base; and
a user interface configured to display the ranging data.

18. The ranging beacon system of claim 17, wherein the computing device is a smartphone, a tablet, a laptop, or a desktop computer.

19. The ranging beacon system of claim 17, wherein the computing device comprises a processor configured to determine at least one of run time, speed, acceleration, reaction time, force-velocity, step rate, step index, torso angle, step count, step length, leg turnover, ground contact time (GCT), flight time, vertical oscillation, step speed loss, maximum force (Fmax), and spinout speed based on the ranging data.

20. The ranging beacon system of claim 19, wherein the user interface is configured to display at least one of the run time, speed, acceleration, reaction time, force-velocity, step rate, step index, torso angle, step count, step length, leg turnover, ground contact time (GCT), flight time, vertical oscillation, step speed loss, maximum force (Fmax), and spinout speed based on the ranging data.
